(19) United States
(12) Patent Application Publication
(10) Pub. No.: US 2026/0098239 A1
(43) Pub. Date: Apr. 9, 2026

(54) CELL CULTURE ELECTRIFICATION

(71) Applicant: SYNAXYS, Toulouse (FR)

(72) Inventor: Sophie PAUTOT, Toulouse (FR)

(73) Assignee: SYNAXYS, Toulouse (FR)

(21) Appl. No.: 19/114,129

(22) PCT Filed: Sep. 22, 2023
(86) PCT No.: PCT/EP2023/076275

§ 371 Date: Mar. 21, 2025

(30) Foreign Application Priority Data
Sep. 22, 2022 (EP) 22306401.5

(51) Int. Cl. C12M 1/42; C12M 1/32; C12M 1/34

(52) U.S. Cl. CPC C12M 35/02; C12M 23/12; C12M 41/46

(57) ABSTRACT

A three-dimensional cell culture device configured to cultivate cells presenting an electric activity, the culture device includes a plate having at least one well presenting two open extremities, the well being configured to receive at least one cell to be cultivated, a permeable bottom sheet connected to the plate, the bottom sheet closing one open extremity of the well, at least one electrode for measuring the electric activity of the cultivated cells, at least one connection system configured to connect the at least one electrode to an external acquisition system. The cell culture device further includes an electrode support element connected to the plate, configured to carry the at least one electrode, the electrode being configured to extend inside the well.

### FIELD

The present invention relates to three-dimensional (3D) cell culture device.

### BACKGROUND

It is common knowledge that in vitro monitoring of neuronal activity can be carried out by direct or indirect approaches:
indirect approach by means of the observation of chemical reactors/indicator being affected by the neuronal activity,
direct approach by means of electrodes which directly measure the electric neuronal activity.

One of the known indirect approaches is microscopy, which involves a tracer whose physical properties change with the neuronal activity. A well-known example is medical imaging (in vivo) which uses glucose metabolism or oxygen levels inside the blood flows. Medical imaging thus provides macroscopic information (volume of tissue with activity, for example), but no cellular resolution. The resolution of these techniques is therefore not appropriate for in vitro cultures as there is no vascular system to be found in an in vitro culture. Another issue is that the size of the in vitro culture is typically smaller than the spatial resolution of these approaches.

An alternative indirect approach is calcium imaging. This approach allows the measurement of calcium fluctuations occurring during neuronal activity via tracers whose physical properties (for example light emission) change upon calcium binding. With a high-resolution microscope, this approach allows for a good spatial resolution. However, the temporal resolution is slow compared to neurons'spiking activity: it is well-known that the physical switch of the tracer usually takes around 10 milliseconds for the fastest tracer, with a mean switch time around 50 milliseconds, while spiking neuronal events take usually less than 2 milliseconds.

In order to have the best spatial and temporal resolution, there is therefore need of a direct approach with electrodes to measure changes in the electric field potential when neurons are spiking.

Regarding the direct approach, sampling time is comparable with changes in the electrical signal of the measured cells. Electrodes are able to detect changes occurring in a radial distance ranging from 10 μm to 40 μm (depending on the electrodes physical properties). The electrodes of the state of the art are usually microfabricated on a planar support to control their spatial distribution and there are called MEA (Multi Electrodes Array). It is possible to coat the electrodes to get the neurons to grow on them. The performance of the measure thus depends on the distance d between the source (neurons) and the electrodes (detector).

For 2D in vitro cultures, this distance d is minimal as neurons grow directly on the electrodes. However, for 3D in vitro cultures, there are rare physical contacts as the distance between the neurons and electrodes varies with neurons scattered in space. A main issue therefore resides in the fact that the signal pickup by the electrodes of the 2D MEA varies with the distance d of the source (the neurons): when the distance d of the in vitro 3D culture to the electrode increases, the measured signal decreases. This leads to a variability and lack of precisions of the measurements.

In other words, to decrease the distance d, the in vitro 3D culture can be tethered to the MEA via surface treatment to draw a subset of neurons to grow on the MEA. This perturbs the spatial organization of neurons connection with the formation of a 2D expansion of the 3D network. Then, the signal recorded comes from this subset of 2D neuronal network that carries very little information about the 3D neuronal activity of the original network.

To prevent those biases in measurements, 3D in vitro cultures are therefore not tethered to the 2D MEA and are just positioned to rest on their surface. Hence any mechanical shock may lead to dislodge the 3D in vitro cultures away from the surface of the 2D MEA, this leads to additional unreliability in measures.

Another issue with the current recording device for 2D MEA is that they require that the MEA chip is pulled out of the incubator in which the cellular growth takes place to be placed on the recording device for every measurement. This leads to possible damages of the cultivated cell sample.

Finally, in these MEA chips, once the culture has started, cultivated cells from a first culture cannot be moved to be placed in contact with another culture.

There is therefore a need for a solution enabling to:
solve the question of the mechanical stability of the measuring device,
fix the distance between the source and the detector,
enable to measure neuronal activity without damaging the cultivated cell sample,
enabling to connect several cell cultures to each other if needed.

### SUMMARY

The present invention aims at solving this problem and thus relates to a three-dimensional cell culture device configured to cultivate cells presenting an electric activity, said culture device comprising:
a plate comprising at least one well extending parallel to a stacking axis and presenting two open extremities, said well being configured to receive at least one cell to be cultivated,
a permeable bottom sheet configured to retain the at least one cultivated cell while allowing liquid culture medium to flow through and connected to the plate, said bottom sheet closing one open extremity of the well,
at least one electrode for measuring the electric activity of the cultivated cells,
at least one connection system configured to connect the at least one electrode to an external acquisition system,
   wherein the cell culture device further comprises an electrode support element connected to the plate, configured to carry the at least one electrode and to enable said at least one electrode to extend inside the well, said electrode being thus configured to extend inside the well, and
   wherein the connection system is integrated in the plate.

This way, the solution enables to reach the here-above mentioned objective. Especially, it enables to receive cells to assemble 3D neuronal networks, allow their growth while enabling the continuous monitoring of the neuronal activity at fixed positions in the 3D space during the life of the sample while enabling real-time imaging (for example by microscopy).

Furthermore, the cell culture device according to the present invention, enables to move “wired” 3D culture (change their holding place) to place it into contact with another tissue at a desired time without losing the capability to record neuronal activity (insertion on lab on chip device).

The cell culture device according to the invention may comprises one or several of the following features, taken separately from each other or combined with each other:
the electrode support element may be permeable,
the electrode support element may be secured to the plate,
the electrode support element may form the bottom sheet,
the connection system may comprise at least one wire extending inside the plate, the wire presenting two extremities, a first extremity comprising a connection pad configured to cooperate with the electrode and a second extremity comprising a connector configured to connect the external acquisition system,
the plate comprises two plate parts configured to be assembled in an assembled state along respective connection junctions in order to form the well, the electrode support element being secured between the connection junctions of the two plate parts and the connection pad being arranged on at least one of the connection junctions so as to connect with the electrode of the electrode support element,
each connection junction presents an interface surface extending longitudinally along the stacking axis, the interface surfaces of the connection junctions of each plate part contacting each other in the assembled state and the connection pad being arranged on at least one of the interface surfaces,
the three-dimensional cell culture device may comprise at least two electrodes and all the electrodes are carried by the same electrode support element,
the cell culture device may comprise at least two electrodes, and the electrodes are aligned, inside the well, according to a stacking axis of the cell culture device,
the cell culture device may comprise two sets of electrodes and two connections systems, each set of electrodes cooperating with one of the connection systems,
the cell culture device may further comprise a cultivation scaffold configured to fit inside the well and serve as a growth support for the at least one cultivated cell, thus leading to a three-dimensional cell growth,
the cell culture device may comprise a first well and a second well and a first electrode and a second electrode, the first electrode being configured to extend inside the first well, the second electrode being configured to extend inside the second well.

Another object of the present invention concerns a cell cultivation kit comprising:
a three-dimensional cell culture device according to any one of the technical features listed here-above,
an external power source configured to be connected to each connection system,
an external acquisition system configured to collect and store the measured data from the at least one electrode.

The present invention is also about a three-dimensional cell cultivation and activity measuring method carried out by means of the cell culture device and the kit described here-above, the method comprises following steps:
preparing the cell culture device,
placing the cell culture device inside some culture medium,
inserting at least one cultivated cell inside the well,
measuring the electric activity of said at least one cultivated cell.

### BRIEF DESCRIPTION OF THE DRAWINGS

The invention will be better understood, and other aims, details, characteristics and advantages thereof will emerge more clearly on reading the detailed explanatory description which follows, of embodiments of the invention given by way of illustration, purely illustrative and non-limiting examples, with reference to the accompanying drawings:

FIG. 1 is a schematic perspective view of one example of a cultivated cell, a neuron,

FIG. 2 is a view from above of a cell culture without the present invention (left) and with the present invention (right),

FIG. 3 is a schematic axial section of a plate comprising two wells according to the present invention,

FIG. 4 is a view from above of a plate comprising four wells according to the present invention,

FIG. 5 is a schematic perspective view of the cultivation scaffold used for the 3D culture of the cultivated cells inside the wells according to the present invention,

FIG. 6 is a schematic view from above of one well of the present invention,

FIG. 7 is a schematic sectional axial view from one well of the present invention,

FIG. 8 is a schematic sectional axial view of one well comprising an electrode support element according to the present invention,

FIG. 9 is a schematic sectional axial view of one embodiment in which the permeable bottom sheet is the electrode support element,

FIG. 10 is a schematic axial sectional view of the embodiment of FIG. 9,

FIG. 11 is a schematic sectional axial view from one well of another embodiment of the present invention,

FIG. 12 is a schematic sectional axial view of another embodiment a cell culture device 10 configured to cooperate with a lab-on-chip device.

### DETAILED DESCRIPTION

As can be seen on FIG. 2, the present invention is about a three-dimensional cell culture device 10 configured to cultivate cells 100 presenting an electric activity, more particularly cells 100 from the nervous system.

Those cells are part of a culture that might comprise (among other cells and cell types) neurons, astrocytes, oligodendrocytes glial cells and/or microglial cells.

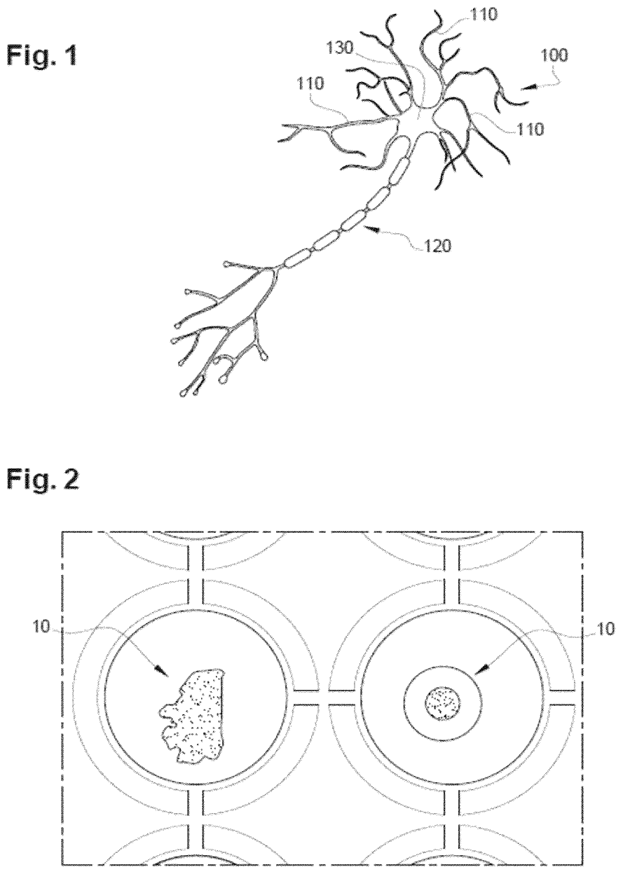

More particularly, the present invention aims at cultivating cells 100 from the nervous system, for example brain cells also called neurons. FIG. 1 depicts an example of cultivated cell 100, a neuron. In the example of the neuron, such a cell 100 displays a particular shape: several dendrites 110 and one axon 120 develop around a central cell body 130 (see FIG. 1) and enable the different neurons to interconnect and communicate with each other by means of electrical and biological messages. Those cells 100 therefore develop by generating a network and connect to each other whenever it is possible. It is therefore important to be able to structure said network construction in order to be able to monitor and understand the precise development mechanisms those cells are involved in. Neurons usually grow and develop in a viable way if they are surrounded and followed in their development by healthy glial cells. Glial cells maintain homeostasis, form myelin in the peripheral nervous system, and provide support and protection for neurons. Glial cells are therefore part of the cultivated cells 100 in the present invention. Their development and growth are equally important than those of the neurons, because neurons cannot survive without them.

In order to correctly measure any electrical information exchanged between several neurons, it is necessary to be able to organize the growth geometry of those neurons. This organization enables scientists to clearly identify the source and the destination of the emitted/received signal. In this regards, FIG. 2 shows the difference between a controlled and structured growth environment (on the right) and a chaotic uncontrolled growth environment (on the left). The structure on the left is very difficult to analyze and cannot lead to strong and solid scientific results. More particularly, in scientific research, repeatability of an experiment is fundamental. A controlled and structured growth environment enables standardization, normalization and minimization of the mechanical perturbation and therefore enables repeatability of the scientific experiment.

Accordingly, the culture device 10 according to the present invention comprises:
a plate 12 comprising at least one well 14 presenting two open extremities, said well 14 being configured to receive at least one cell to be cultivated,
a permeable bottom sheet 16 connected to the plate 12,
at least one electrode 18 for measuring the electric activity of the cultivated cells 100,
at least one connection system 20 configured to electrically connect the at least one electrode 18 to an external acquisition system S.

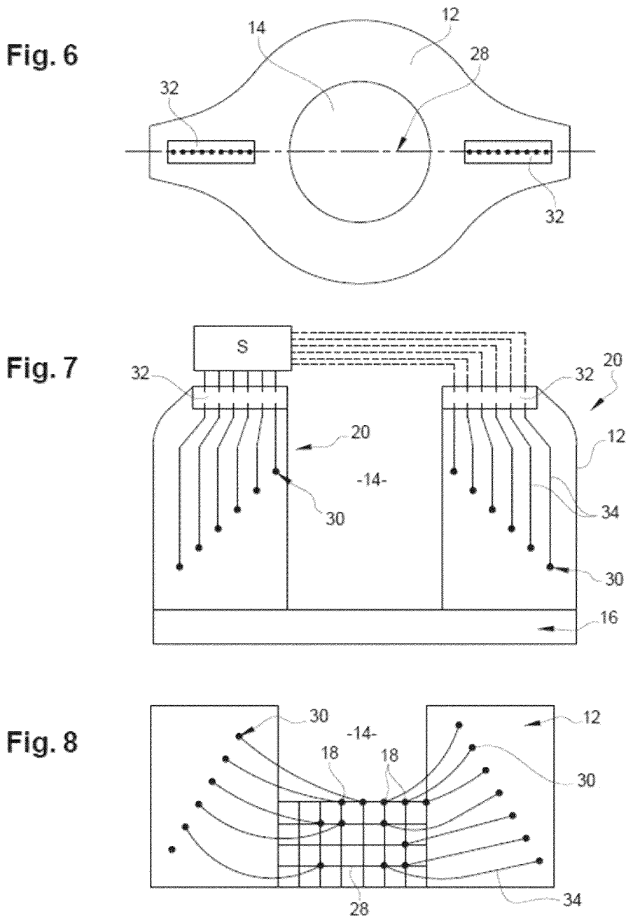

The plate 12 preferably presents a rectangular shape with a side length of about 2 cm. More generally speaking, the present invention uses the terminology “plate” referring to any element presenting two opposite faces which extends transversely with regards to a stacking axis X of the cell culture device 10. More particularly regarding the embodiment of FIGS. 6,7,8, which depicts a single-well configuration, hence the plate 12 preferably reproduces the geometry of the well 14, and might thus display other shapes than a rectangular shape. The width of the plate 12 is preferably of less than 3 cm (preferably comprised between 1 and 3cm) in order to fit in standard culture plates. In some embodiments, the width of the plate 12 is preferably of less than 8 mm in order to fit in a standard tube, such as an Eppendorf tube®. This enables an easy and safe handling of the cell culture device 10 without using too much material. The plate 12 is preferably made of transparent material in order to facilitate cells observation through it. This eases the observation steps, as an operator simply needs to transfer the cell culture device 10 on a magnifier or a microscope to enable a clear observation of the growing and development of the cultivated cells 100. The plate 12 can for example be made of Polydimethylsiloxane. Preferably, the used material for the plate 12 is cell culture compatible and easy to sterilize and to handle in order to allow an easy and quick production.

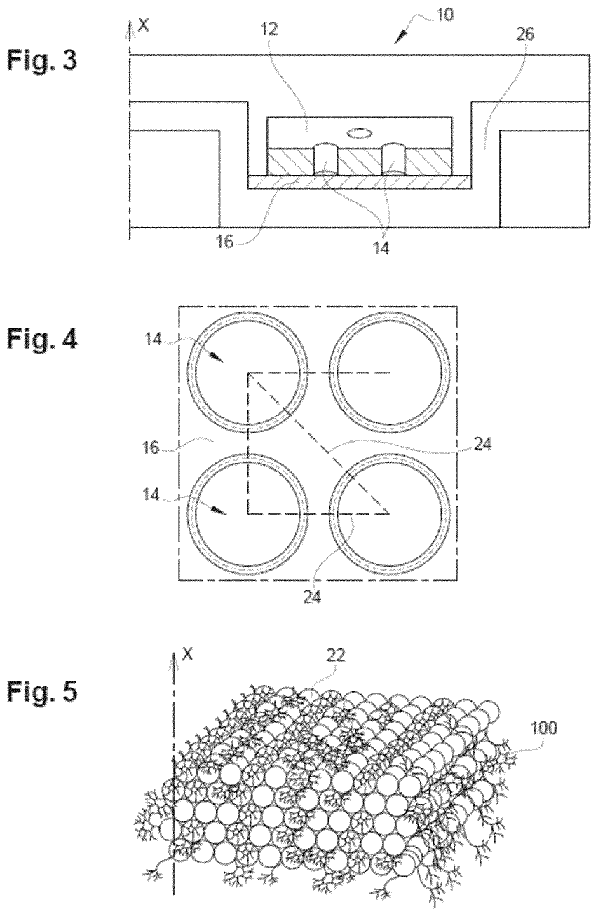

As already mentioned, and as can be seen on FIG. 3a, the plate 12 comprises at least one well 14. According to the present invention, the well 14 extends along the stacking axis X of the cell culture device 10. More particularly, the well 14 extends between two opposite extremities each one opening in one surface of the plate 12. The well 14 configured to receive at least one cell 100 to be cultivated. All cultivated cells from each well 14 are thus cultivated in the same culture medium. In some cases, when the plate 12 comprises several wells 14, culture conditions might change from one well 14 to another, by adding perturbing elements inside a defined well 14. Such a perturbing element can for example be a foreign cell, like a tumorous cell, for example. In order to offer sufficient culture medium to the cultivated cells 100, each well 14 displays a diameter that ranges from 1 mm to 8 mm. The routine standard of the present invention is of 2 mm of diameter. Each well 14 displays a height, along the stacking axis X of maximal 4 mm. The well 14 presents one free opening and one closed opening. The closed opening is preferably located on the bottom surface according to the stacking axis X. The closed opening is closed by the permeable bottom sheet 16. The well 14 may present a cylindrical or a conical shape.

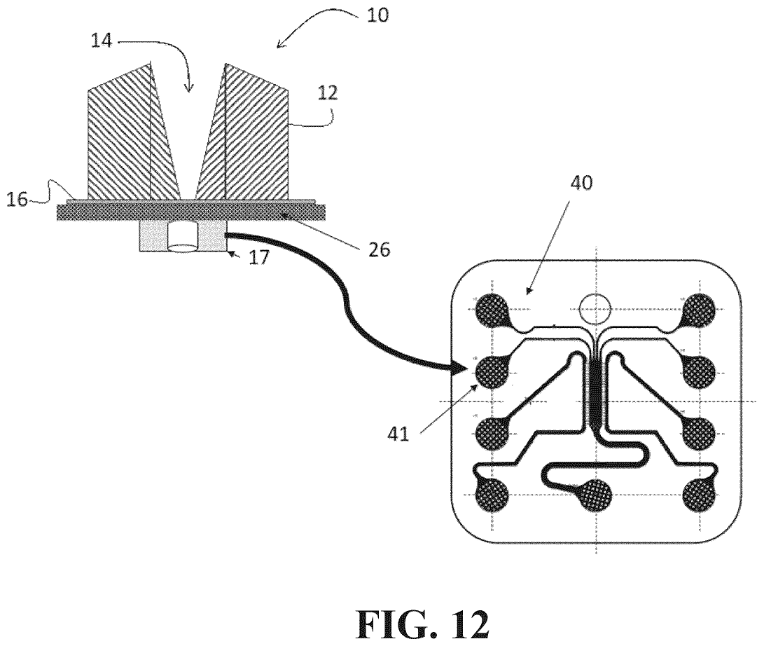

In case the well 14 presents a conical shape as illustrated in FIG. 12, its diameter increases along the stacking axis X starting from the bottom surface (from the permeable bottom sheet 16). In such embodiment, the free opening presents a larger diameter than the closed opening. The free opening of the well 14 may also present rounded or beveled edges. Regarding the very small dimensions of the well 14, a conical shape and/or rounded or beveled edges enable to change the wetting angle of the culture medium to the surface of well 14. Thus a surface tension allows the formation of the liquid drop slightly taller than the height of well 14 which enables the maximization of the medium contained by well 14. This effect could be further increased by a surface treatment of the edges. This enables to make sure that all cultivated cells 100 are bathed inside the culture fluid. As the cultivated cells 100 form a network and thus a tissue, the size of the formed tissue depends on the size of the wells 14.

One example of medium are saline solutions supplemented with amino acids, vitamins, growth factors and glucose optimized for the cells grown in the device

In case of neuronal cell culture (and any other cell from the nervous system), it is important to adapt the growing environment. Therefore, the cell culture device 10 comprises a cultivation scaffold 22 configured to fit inside the well 14. This cultivation scaffold 22 extends along the stacking axis X of the cell culture device 10. In the embodiment depicted, the cultivation scaffold 22 is preferably a series of cultivation beads 22 configured to fit inside the well 14. Those cultivation beads 22 are made of glass or gel and typically measure 30 to 100 μm. The cultivation beads 22 are stacked up inside the well 14 along the stacking axis X, as can be seen on FIG. 5. The cultivation beads 20 serve as a growth support for the cultivated cells 100. The well 14 is thus configured to enable a three-dimensional cell growth. As mentioned above, those three-dimensional growth is important to ensure a reasonable longevity of neuronal cells (and any other cell type from the nervous system) which are not keen growing and developing in one dimensional environment.

The cell culture device 10 comprises at least one well 14. This embodiment of the cell culture device 10 is conceived to fit inside a tube, such as an Eppendorf E tube®. In order to study and observe the interaction and communication of cultivated cells 100, especially in case of neurons, the plate 12 comprises at least two wells 14. Preferably, one single plate 12 may comprise 1 to 24 wells 14.

As can be seen on FIG. 3, the plate 12 is secured on the bottom sheet 16 following the stacking axis X. The bottom sheet 16 thus closes one open extremity of each well 14 of the plate 12. In other words, the bottom sheet 16 is configured to retain the cultivated cells 100 inside their well 14 while allowing liquid culture medium to flow through. The bottom sheet 16 is therefore a permeable sheet. The permeability of the bottom sheet 16 should be comprised between 10 μm and 100 μm. The permeable bottom sheet 16 is preferably made of a flexible and/or soft material which can be deformed under a user's pressure. Such a deformation might help to pull any cultivated cell 100 out of the well 14 if needed. The bottom sheet 16 is further preferably configured to enable a retrieval of a sample portion of the bottom sheet 16 where cultivated cells are localized to be easily separated from a remaining portion of the bottom sheet 16. The sample portion of the bottom sheet 16 can be cut, punched or separated in any other suitable manner from the remaining portion. For example, as shown in FIG. 3, the bottom sheet 16 may comprise a meshed structure with meshes having a size smaller than the size of the cultivation beads 22 (or any other cultivation scaffold 22) while enabling the liquid culture medium to circulate through and feed the cultivated cells 100 inside (or outside) the well 14. The cultivated cells together with the sample portion of the bottom sheet 16 can be collected by means of a biopsy punch.

In order to put two wells 14 in communication, and to measure the communication between two different cells 100 cultures, the bottom sheet 16 might comprise two layers: a bottom layer and a top layer (regarding the stacking axis X). In those embodiments, the bottom layer serves as a support layer, supporting and closing the cell culture device 10, and the top layer of the bottom sheet 16 serves as a “communication layer” in which the cultivated cells 100 can move following pre-designed growing paths 24. This top layer can for example be a glue layer connecting the bottom layer of the bottom sheet 16 to the plate 12, in which some growing paths 24 have been sculpted in order to put two wells 14 in communication (see FIG. 4). The growing paths 24 comprise at least one opening connecting one well 14 to another. Each opening extends preferably transversally with regards to the stacking axis X of the cell culture device 10. Such an opening can present a length comprised between 10 μm and 500 μm.

In order to enable an easy observation of the cultivated cells 100 through the cell culture device 10, additionally to the plate 12, the bottom sheet 16 is also preferably made of a transparent material.

As can be seen on FIG. 3, the cell culture device 10 according to the present invention further comprises a support piece 26 configured to receive one or several cell culture devices 10 in order to enable a safe and easy displacement of the cell culture devices 10.

This support piece 26 further enables the plate 12 to be safely completely immerged inside a culture medium filling a culture dish, without squeezing or stretching the bottom sheet 16 and/or damaging the wells 14 and thus the cultivated cells 100 (see FIG. 3). The support piece 26 can be porous or comprise apertures, in order to enable the culture medium to circulate freely. Said support piece 26 further offers the possibility to change cell culture medium at once by lifting the cell culture device 10 up and placing it in a new culture dish containing new culture medium. The support piece 26 may have centering elements configured to center the support piece 26 with respect to the culture dish.

As the plate 12 is completely immerged in the liquid culture medium, the electrical activity of the cultivated cells 100 can be easily monitored by at least one electrode 18 (see FIG. 3).

As already mentioned above, it is well known that neuron cells communicate with each other using neurotransmitters which induce, along the axons 120, an electrical potential modification around the axon 120 membrane and thus generate an electrical signal which spreads along said axon 120 and reaches the next cell 100. This electrical potential modification around the axon 120 membrane induces changes inside the electrical field around the cells 100, inside the well 14, and those changes can be measured by the electrodes 18.

Measures can be taken inside a single well 14 between different cultivated cells 100 of the same well 14 or, according of the embodiments, measures can be taken between two (or more) wells 14 from cultivated cells 100 growing in different wells 14. In those cases, as the length of the axons 120 are known thanks to the openings of the growing paths 24 of the bottom sheet 16, one has:
a signal source (cell 100 from first well 14)
a signal destination (cell 100 from second well 14)
a known signal path length (length of the openings of the growing path 24 between the two wells 14).
   With all those known parameters, an information propagation speed can thus easily be calculated.

Therefore, as can be seen on FIGS. 7 and 8, the cell culture device 10 further comprises, inside each well 14, at least one electrode 18. More precisely, each electrode 18 is configured to extend inside their associated well 14.

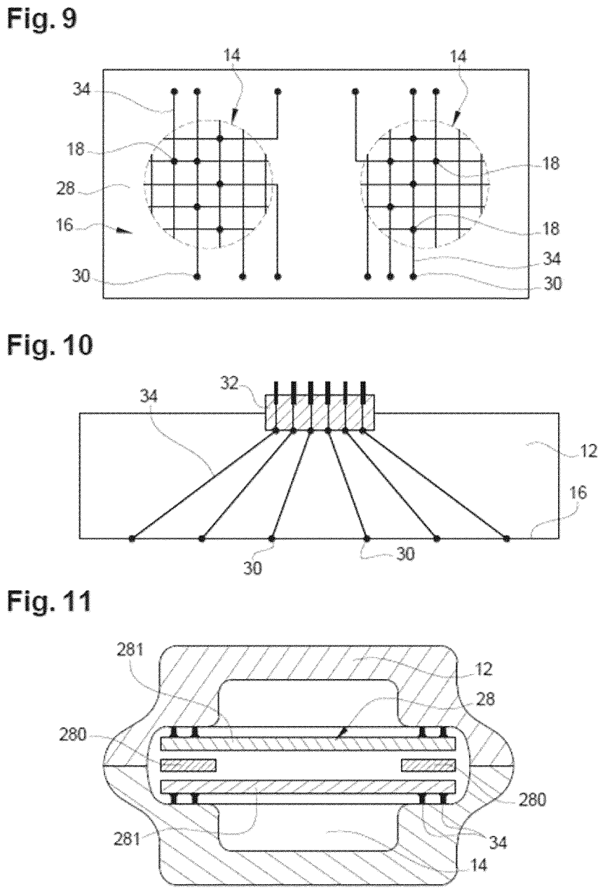

As can be seen on FIGS. 7 and 9, the cell culture device 10 according to the present invention further comprises an electrode support element 28 connected to the plate 12. In FIG. 9, the support element 28 is the bottom sheet 16. Preferably, the electrode support element 28 is secured to the plate 12. This electrode support element 28 is configured to carry the at least one electrode 18 and to enable said at least one electrode 18 to extend inside the well 14. This enables each electrode 18 to be maintained at a known and predefined distance from the edge of the well 14. As this support element 28 preferably extends inside the well 14, the electrode support element 28 is preferably a permeable support element 28 in order to minimize its impact on culture media flow and cultivated cells 100 growth and communication. In this regard, the electrode support element 28 is preferably flexible.

According to the embodiment depicted on FIGS. 6 and 7, the electrode support element 28 is a permeable flexible sheet secured vertically (regarding the stacking axis X) inside the well 14, preferably in its middle. The permeable flexible sheet could be made, at least partly, from ceramic with metallic deposition. In a further embodiment, depicted on FIG. 11, the electrode support element 28 is an ensemble of flexible sheets 281 separated from each other by at least one spacer 281. This ensemble of flexible sheets 281 and spacers 280 forms a sort of vertical honey comb or racking structure extending along the stacking axis X of the cell culture device 10.

In another not represented embodiment, the electrode support element 28 could be a tree extending from the center of the well 14 along the stacking axis X of the cell culture device 10.

In another embodiment (see FIGS. 9 and 10), the electrode support element 28 is the bottom sheet 16.

As shown on FIGS. 7 and 8, each well 14 comprises at least two electrodes 18 configured to communicate with each other, thus enabling a precise monitoring of the electrical communication of the cultivated cells 100. Therefore, according to this embodiment, the cell culture device 10 according to the present invention comprises at least two electrodes 18 and all the electrodes 18 are carried by the same electrode support element 28. In the case of the embodiment of FIG. 7, all the electrodes are carried by the permeable flexible sheet 16 secured in the middle of the well 14. This minimizes the steric discomfort for the cultivated cells 100. It also enables a simple structure and an easy fix in case one of the electrodes 18 had to be changed. Regarding the embodiments, it might not be possible to change a single electrode 18, however, changing the electrode support element 28 is also advantageous in comparison of changing the whole cell culture device 10.

It is important to note that once the in vitro culture has started, the electrodes 18 cannot be changed without destroying said in vitro culture. However, in the embodiments in which the plate 12 can be detached, the sample can be recovered with the electrodes 18 for further analysis, and each plate 12 can be reused with a new support element 28.

Such a central and single electrode support element 28 also enables to conceive a cell culture device 10 that is dismountable, and to conceive each well 14 as a two pieces element which can be easily assembled. Therefore, the plate 12 comprises preferably two plate parts, possible symmetrical, configured to be assembled in an assembled state in order to form the well 14. This eases industrialization of said cell culture device 10. More generally speaking, the plate 10 may comprise two distinct plate parts configured to be assembled in order to form the plate 12. Those two distinct plate parts are further configured to be assembled in order to form the well 14, the end result being symmetric (or not) independently of the potential symmetry of the different parts.

The possibility to assemble the complete cell culture device 10 from several parts, including plate parts, further enables to easily secure the electrode support element 28 to the plate 12 while the cell culture device 10 is assembled. In the cases wherein the plate 12 is made of two plate parts to be assembled together, each of them presents a connection junction along which the plate parts are joined. Each connection junction presents an interface surface contacting the interface surface of the other connecting junction in the assembled state. In the represented embodiment, the interface surfaces extend longitudinally, namely parallel to the stacking axis X.

In those cases, the electrode support element 28 is secured between the connection junction of the two plate parts. This enables an easy assembling and disassembling of the cell culture device 10.

As also depicted on the embodiment of FIG. 7, all the electrodes 18 are preferably aligned, inside the well 14, according to the stacking axis X of the cell culture device 10.

The advantages of such a vertical alignment along the stacking axis X of the cell culture device 10 is preferable to a horizontal alignment as a horizontal alignment could lead to difficulties to add or remove cultivated cells or culture media or disturb the 3D growth of the cultivated cells along the cultivation scaffold 22.

In order to process the information collected by the electrodes 18, in each well 14, the cell culture device 10 further comprises as already mentioned, at least one connection system 20 configured to electrically connect the at least one electrode 18 to an external acquisition system S.

This external acquisition system is preferably a standard acquisition system, commonly available by any person skilled in the art of data acquisition. Said external acquisition system might comprise several modules, one for each set of electrodes. The usual acquisition systems can support a maximal of 32 electrodes, hence to maximize the number of recorded electrodes it could be necessary to use multiple elements.

In order to protect the connection system 20, and to avoid any kind of shifting and destruction it is preferably integrated in the plate 12, as can be seen on FIGS. 6 and 7. In this embodiment, the connection system 20 comprises a series of connection pads 30 integrated inside the plate 12 and an external socket 32. Each connection pad 30 is electrically connected to at least one electrode 18 and to the external socket 32. The connection pads 30 thus form an electrical connection interface between the electrodes 18, located on the electrode support element 28, the rest of the connection system 20 integrated in the plate 12, and the external socket 32.

This enables the connection system 20 to be integrated in the plate 12 while enabling a removal of the support element 28 and the electrodes, without having to discard the complete plate 12. In case the plate 12 is made of several plate parts to be assembled in order to form the wells 14, the connection pads 30 are located on at least one of the interface surfaces of the connection junctions. This is illustrated on FIGS. 7 and 8. This precise positioning at the connection junction(s) enables an easy electrical connection with the corresponding electrodes 18 of the electrode support element 28 while the cell culture device 10 is assembled. When the electrode support element 28 is secured between the interface surfaces of the plate parts, each electrode 18 is electrically connected to its corresponding connection pad 30.

The external socket 32 is preferably situated on the cell culture device 10, on an external surface of the plate 12. Preferably, the external socket 32 is situated on the upper surface of the plate 12 (according to the stacking axis X). However, any technically relevant location of said external socket 32 could be possible. The external socket 32 is configured to be connected to the external acquisition system S. This electrical connection can for example be realized by Wi-Fi. The advantage of coupling the external acquisition system S with Wi-Fi to the cell culture device 10 allows to keep the cell culture device 10 in the incubator and to pilot the data measuring and recording remotely.

Regarding the high number of small elements comprised in the connection system 20, such an integration inside the plate 12 further enables to secure each element in a specific place from which it cannot break away and this ensures that all the mechanical parameters of the cell culture device 10 are known and perfectly set. This improves greatly the scientific repeatability of any experience carried out by means of the present cell culture device 10.

More particularly according this embodiment, the connection system 20 comprises at least one wire extending inside the plate 12 (more particularly one wire 34 per integrated connector 30). More generally speaking, the connection system 20 could comprise any sort of electrical connecting element, like for example a coating or a layer. It could be printed circuits, for example. By means of simplification, the term “wire” is to be understood in a very broad technical sense. However, among all those possible electrical connecting elements, cable wires are the preferred version. Each wire 34 presents two extremities, a first extremity comprising the connection pad 30 configured to cooperate with the electrode 18 and a second extremity comprising a connector configured to connect the external socket 32 and thus the external acquisition system S. Once the electrode support element 28 is assembled with the plate 12, each connection pad 30 is electrically connected to its corresponding electrode(s) and each electrode 18 is thus connected to the external socket 32.

Regarding the particular embodiment of FIGS. 6, 7 and 10 each wire 34 electrically connecting the connection pad 30 to the external socket 32 extends, inside the plate 12, for example parallel to the stacking axis X of the cell culture device 10. On the other hand, each wire 34 connecting each connection pad 30 to their associated electrodes 18 extends along the electrode support device 28, preferably in a direction globally perpendicular or at least intersecting the stacking axis X. The wire 34 connecting the external socket 32 to the connection pads 30 extend thus in a different direction than the wires connecting the electrodes 18 to the connection pads 30. The connection pads 30 thus form an angle in the electronic circuit of the connection system 20.

The length of each wire 34 (and thus the spatial positioning of the electrodes 18) has to be minimal to prevent perturbations of the measurement by artefacts due to mechanical and electrical perturbations.

As can be seen on FIGS. 6, 7 and 8, the cell culture device 10 comprises two sets of electrodes 18 and two connections systems 20, each set of electrodes cooperating with one of the connection systems 20. This enables to pass more wires, and thus allow more electrodes (recording points).

As already mentioned above, some embodiments of the present invention enable to create cellular communication between two cultures of two distinct wells 14. In those embodiments, the cell culture device 10 comprises a first well 14 and a second well 14 and a first electrode 18 and a second electrode 18 (see FIG. 9), the first electrode 18 being configured to extend inside the first well 14, the second electrode 18 being configured to extend inside the second well 18. All the electrodes 18 are carried by the same electrode support element, regardless of the connection system 20 to which they belong. This is also possible with more than two wells 14, depending on the embodiments.

In order to ease the accessibility of the scientific measurements for any kind of laboratory and/or operator, the cell culture device 10 according to the present invention might be part of a cell cultivation kit comprising:
a three-dimensional cell culture device 10 as described here-above,
an external power source (not represented) configured to be connected to each connection system 20,
an external acquisition system S configured to collect and store the measured data from the at least one electrode 18.

The cell culture device 10 according to the present inventions enables the implementation of a cultivation method which comprises the following steps:
all the parts of the cell culture device 10 are cleaned and sterilized with the appropriate method prior to assemble,
if guiding elements are required, a coating of poly-Lysine is applied on the bottom sheet 16 to prime its surface before the deposition of an active compound (for example AMPc) working as a signalling system which induces and/or enhances or limits/stops cell growth,
assembling of the different elements of the cell culture device 10: the electrode support element 28 is positioned to interface the connecting elements 30 and, according to the embodiment on FIGS. 6,7,8, pinched between the two halves of the plate 12 until the adhesive layer is set,
once the cell culture device 10 is assembled and dried, the cell culture device 10 is immersed in ethanol for 30 min and further dried under sterile conditions,
placing the cell culture device 10 in a humidity chamber overnight in order to facilitate the wetting of all the elements and decrease the surface tension for preventing the formation of air bubble (in particular in front of the electrodes 18),
immersing the cell culture device 10 in a saline solution, and checking that there are no air bubbles where cells are supposed to grow,
replacing the saline solution replaced by a small volume of cell culture medium; in order to prevent drying and allowing better visualization of the compartment that will receive cultivated cells,
pipetting colloidal cell culture (between two and six days old, the quantity of pipetted cells depending on the experiment to be carried out) from its starting culture dish into the well 14 of the cell culture device 10,
completing medium to insure sufficient nutrition for several days,
putting the cell culture device 10 inside an incubator,
when the activity of the cultivated cells sample inside the well 14 needs to be recorded; pulling the sample from the incubator and connecting the cell culture device 10 to the external acquisition system S,
recording the measured data by the electrodes 18,
recording of the neuronal activity in synchronization with the addition of an active agents to the culture medium using a perfusion device,
processing the Data offline by using home designed software.

The described method also offers the possibility of treating the cultivated cells 100 with foreign agent (chemical compound, virus, foreign cells, for example) before measuring their electronic activity.

The present invention thus enables to prevent measure bias by presenting a growth support that does not perturb 3D neuronal network formation and cell growth. The present invention makes it possible to produce a cell culture device 10 with at least one electrode 18 enabling each electrode 18 to be connected with the connection system 20 and the acquisition system S. The present cell culture device 10 further enables each electrode 18 and their connections to be protected from the humidity of the cell culture well 14. Each connection is therefore designed according to the constraints of the culture and does not interfere with the culture processes.

The elements of the cell culture device 10 according to the present invention is easy to sterilize, easy to assemble, and easy to connect to the external acquisition system S (the cell culture device 10 can be plugged and unplugged without stressing the cell culture inside the well 14), and offers space to load cultivated cells and permit 3D cell growth and further cell observation by microscopy. Depending on the embodiment, data can also be collected and analysed remotely, without interfering with the cells 100.

The fact that the permeable bottom sheet 16 is permeable offers the possibility to put the cultivated cells 100 network in contacts with other cell type, for example by plugging them on an existing lab-on-chip device 40 while being pre-wiring to record the cultivated cells 100 activity. In this respect, FIG. 12 shows a cell culture device 10 having protrusion 17 extending from a bottom of the support piece 26 along the stacking axis X, opposite the plate 12, and provided with a bore in communication with the well 14. The protrusion 17 is configured to be fitted in an inlet 41 of the lab-on-chip device 40 to enable the cultivated cells in contact with any other suitable tissue type.

The present device 10 also offers the possibility to take apart the system at the end of the experiment to recover the sample of cultivated cells for further analysis.

1-13. (canceled)

14. A three-dimensional cell culture device configured to cultivate cells presenting an electric activity, said culture device comprising:
a plate comprising at least one well extending parallel to a stacking axis (X) and presenting two open extremities, said well being configured to receive at least one cell to be cultivated,
a permeable bottom sheet configured to retain the at least one cultivated cell while allowing liquid culture medium to flow through and connected to the plate, said bottom sheet closing one open extremity of the well,
at least one electrode for measuring the electric activity of the cultivated cells,
at least one connection system configured to connect the at least one electrode to an external acquisition system,
wherein the cell culture device further comprises an electrode support element connected to the plate, configured to carry the at least one electrode and to enable said at least one electrode to extend inside the well, said electrode being thus configured to extend inside the well,
wherein the connection system is integrated in the plate.

15. The three-dimensional cell culture device according to claim 14, wherein the electrode support element is permeable.

16. The three-dimensional cell culture device according to claim 14, wherein the electrode support element is secured to the plate.

17. The three-dimensional cell culture device according to claim 14, wherein the electrode support element forms the bottom sheet.

18. The three-dimensional cell culture device according to claim 14, wherein the connection system comprises at least one wire extending inside the plate, the wire presenting two extremities, a first extremity comprising a connection pad configured to be electrically connected to the electrode and a second extremity comprising a connector arranged on an outer surface of the plate and configured to be electrically connected to the external acquisition system(S).

19. The three-dimensional cell culture device according to claim 18, wherein the plate comprises two plate parts configured to be assembled in an assembled state along respective connection junctions in order to form the well, the electrode support element being secured between the connection junctions of the two plate parts and the connection pad being arranged on at least one of the connection junctions so as to connect with the electrode of the electrode support element.

20. The three-dimensional cell culture device according to claim 19, wherein each connection junction presents an interface surface extending longitudinally along the stacking axis (X), the interface surfaces of the connection junctions of each plate part contacting each other in the assembled state and the connection pad being arranged on at least one of the interface surfaces.

21. The three-dimensional cell culture device according to claim 14,
wherein the three-dimensional cell culture device comprises at least two electrodes, and
wherein all the electrodes are carried by the same electrode support element.

22. The three-dimensional cell-culture device according to claim 21,
wherein the cell culture device comprises at least two electrodes, and
wherein the electrodes are aligned, inside the well, according to the stacking axis (X).

23. The three-dimensional cell-culture device according to claim 21, wherein the cell culture device comprises two sets of electrodes and two connections systems, each set of electrodes cooperating with one of the connection systems.

24. The three-dimensional cell culture device according to claim 14, wherein the cell culture device comprises a first well and a second well and a first electrode and a second electrode, the first electrode being configured to extend inside the first well, the second electrode being configured to extend inside the second well.

25. A Cell cultivation kit comprising:
a three-dimensional cell-culture device according to claim 14,
an external power source configured to be connected to each connection system,
an external acquisition system(S) configured to collect and store the measured data from the at least one electrode.

26. A three-dimensional cell cultivation and activity measuring method carried out by means of the cell culture device according to claim 14 and a cell cultivation kit, wherein the method comprises following steps:
preparing the cell culture device,
placing the cell culture device inside some culture medium,
inserting at least one cultivated cell inside the well,
measuring the electric activity of said at least one cultivated cell.
